(19) United States
(12) Patent Application Publication
(10) Pub. No.: US 2024/0078665 A1
(43) Pub. Date: Mar. 7, 2024

(54) SYSTEM AND METHOD FOR PREDICTING METASTATIC PROPENSITY OF A TUMOR

(71) Applicant: RAMBAM MED-TECH LTD., Haifa (IL)

(72) Inventor: Myroslav Lutsyk, Migdal HaEmek (IL)

(73) Assignee: RAMBAM MED-TECH LTD., Haifa (IL)

(21) Appl. No.: 18/241,502

(22) Filed: Sep. 1, 2023

Related U.S. Application Data
(60) Provisional application No. 63/403,003, filed on Sep. 1, 2022.

(51) Int. Cl. G06T 7/00; G16H 50/20

(52) U.S. Cl. CPC G06T 7/0012; G16H 50/20; G06T 2207/10072; G06T 2207/20081; G06T 2207/30096

(57) ABSTRACT

A system and method of predicting propensity of metastasis of a tumor in a patient by at least one processor may include: receiving, from a first scan modality, a first scan comprising a set of scan images depicting metabolic information; receiving, from a second scan modality, a second scan comprising a set of scan images depicting anatomical information; segmenting the first scan to identify a volumetric segment representing a suspected tumor, based on the depicted metabolic information; extracting one or more radiomics features from the second scan, corresponding to the volumetric segment, based on the depicted anatomical information; and predicting propensity of metastasis of the suspected tumor, based on the one or more radiomics features.

### CROSS-REFERENCE TO RELATED APPLICATIONS

This application claims the benefit of priority of U.S. Patent Application No. 63/403,003, filed Sep. 1, 2022, which is hereby incorporated by reference in its entirety.

### FIELD OF THE INVENTION

The present invention relates generally to diagnostic technology. More specifically, the present invention relates to systems and methods of predicting metastatic propensity of a tumor.

### BACKGROUND OF THE INVENTION

In recent years, prostate cancer has emerged as a prevalent form of cancer among middle-aged, and elder male patients. Although mortality rates among prostate cancer patients are relatively low in relation to other, more aggressive forms of cancer, there is importance in identifying a propensity for metastasis for determining treatment, or management of the disease.

Currently available methods of assessing propensity of metastasis in prostate cancer patients include various empiric formulae, such as the Roach equation, which are notoriously inaccurate, or overly dependent on the professional and diagnostic capabilities of the treating physician or radiologist.

### SUMMARY OF THE INVENTION

Embodiments of the invention may provide a practical application for accurately determining propensity of cancer metastasis, in a manner that is devoid of human error. Embodiments may thus include an improvement over currently available technology of assistive diagnostics.

Additionally, and as elaborated herein, embodiments of the invention may include specific algorithms for intelligently selecting a unique combination of metabolic and radiomic features, obtained from two or more scanning modalities. As shown herein (e.g., in relation to FIG. 4), this selected combination of features may provide the prediction of metastasis propensity with optimal performance (e.g., accuracy) metrics.

Embodiments of the invention may include a method of predicting propensity of metastasis of a tumor in a patient by at least one processor. Embodiments of the method may include receiving, from a first scan modality, a first scan that includes a set of scan images depicting metabolic information; receiving, from a second scan modality, a second scan that includes a set of scan images depicting anatomical information; segmenting the first scan to identify a volumetric segment representing a suspected tumor, based on the depicted metabolic information; extracting one or more radiomics features from the second scan, corresponding to the volumetric segment, based on the depicted anatomical information; and predicting propensity of metastasis of the suspected tumor, based on the one or more radiomics features.

For example, the volumetric segment may be, or may be included in a prostate of the patient, and wherein the metabolic information represents a depicted intake of Prostate-Specific Membrane Antigen (PSMA) within the prostate.

Additionally, or alternatively, the at least one processor may determine a prognosis and/or a suggested course of treatment for the suspected tumor, based on the predicted propensity of metastasis.

In some embodiments, the second scan modality may be a Positron Emission Tomography (PET) scan modality, and the first scan modality may be, for example a Computed Tomography (CT) scan modality, a Magnetic Resonance Imaging (MRI) scan modality, and the like.

According to some embodiments, the at least one processor may predict propensity of metastasis of the suspected tumor by applying at least one machine-learning (ML) model on the extracted radiomics features, to produce the prediction of propensity of metastasis.

For example, the at least one processor may extract, from the volumetric segment of the first scan, at least one metabolic feature representing the depicted metabolic information, and train the at least one ML model to produce the prediction of propensity of metastasis, based on (a) the radiomics features and/or (b) the at least one metabolic feature.

According to some embodiments, the at least one processor may apply a feature selection algorithm on a group of features that includes (a) the radiomics features and/or (b) the at least one metabolic feature. The at least one processor may thus select a subset of the group of features, based on the propensity of metastasis as predicted by the at least one ML model. The at least one processor may subsequently further train the at least one ML model based on the subset of the group of features, to produce the prediction of propensity of metastasis.

Additionally, or alternatively, the at least one processor may train the at least one ML model in an iterative process. In such embodiments, at least one iteration of the iterative process may include: receiving a first group of features selected from (a) the radiomics features and (b) the at least one metabolic feature; selecting a second group of features that may be a subset of the first group; training the at least one ML model to produce a prediction of propensity of metastasis, based on the second group of features; and providing the second group of features as a first group of features for a subsequent iteration, based on propensity of metastasis as predicted by the at least one ML model.

According to some embodiments, the at least one processor may extract at least one metabolic feature, representing the metabolic information depicted in the volumetric segment of the first scan, and receive annotation data, representing propensity of metastasis, corresponding to at least one of the first scan and second scan. The at least one processor may subsequently train the at least one ML model to produce a prediction of propensity of metastasis, based on at least one of the extracted radiomics features and the at least one metabolic feature, according to the annotation data.

According to some embodiments, the at least one processor may extract the radiomics features by computing a Grey Level Co-occurrence Matrix (GLCM), based on at least one image of the second scan; and calculating at least one GLCM-based radiomics feature based on the GLCM matrix. In such embodiments, the at least one GLCM-based radiomics feature may include a GLCM joint entropy feature, a GLCM joint energy feature, a GLCM difference entropy feature, a GLCM contrast feature, a GLCM sum squares feature, a GLCM difference average feature, a GLCM Inverse Difference feature, a GLCM Inverse Difference Moment IDM feature, and any combination thereof.

Additionally, or alternatively, the at least one processor may extract the radiomics features by applying one or more wavelet-based algorithms on the GLCM matrix, to produce at least one wavelet-based GLCM radiomics feature. The at least one wavelet-based GLCM radiomics feature may include, for example an HLL (High-Low-Low) GLCM joint entropy feature, an HLL GLCM difference entropy feature, and an HLL GLCM sum entropy feature.

Additionally, or alternatively, the at least one processor may extract the radiomics features by computing a Grey Level Run Length Matrix (GLRLM), based on at least one image of the second scan; and calculating a Normalized Gray Level Non-uniformity (GLNN) radiomics feature based on the GLRLM matrix.

Additionally, or alternatively, the at least one processor may extract the radiomics features by applying one or more wavelet-based algorithms on the GLRLM matrix, to produce at least one wavelet-based GLRLM radiomics feature. The wavelet-based GLRLM radiomics feature may include, for example an HLL GLRLM Normalized Gray Level Non-uniformity feature, an HLH (High-Low-High) GLRLM Short Run Emphasis feature, an HLH GLRLM Short Run High Gray Level Emphasis feature, an HLH GLRLM Long Run Low Gray Level Emphasis feature, an HLH GLRLM Run Entropy feature, and any combination thereof.

Additionally, or alternatively, the at least one processor may extract the radiomics features by computing a wavelet-based radiomics feature, based on at least one image of the second scan. The wavelet-based radiomics feature may include, for example an HLL first order median feature, an HLL first order Robust Mean Absolute Deviation feature, an HLL first order Mean Absolute Deviation feature, an HLL first order entropy feature, an HLL first order X-Percentile feature, and an LLL (Low-Low-Low) first-order uniformity feature.

Additionally, or alternatively, the at least one processor may extract the radiomics features by computing a Grey Level Dependence Matrix (GLDM), based on at least one image of the second scan; and calculating at least one GLDM-based radiomics feature based on the GLDM matrix. The GLDM-based radiomics feature may include, for example a GLDM grey level variance feature.

Additionally, or alternatively, the at least one processor may apply one or more wavelet-based algorithms on the calculated GLDM matrix, to produce corresponding wavelet-based GLDM radiomics features, including for example an LLH (Low-Low-High) GLDM Small Dependence High Gray Level Emphasis feature, an HLH GLDM Small Dependence High Gray Level Emphasis feature, and an LLH GLDM Small Dependence Low Gray Level Emphasis feature.

According to some embodiments, the at least one ML model may include a first ML model that is a binary classifier, and a second ML model that is a random forest ML model.

In such embodiments, both the binary classifiers and the random forest ML models may be trained to produce a prediction of propensity of metastasis, based on at least one of: (a) the radiomics features and (b) the at least one metabolic feature. The at least one processor may subsequently arbitrate between prediction of propensity of metastasis of both ML models, and produce a prediction (e.g., a unified prediction) of propensity of metastasis of the suspected tumor, based on the arbitration.

Embodiments of the invention may include a system for predicting propensity of metastasis of a tumor. Embodiments of the system may include a non-transitory memory device, wherein modules of instruction code may be stored, and at least one processor associated with the memory device, and configured to execute the modules of instruction code.

Upon execution of said modules of instruction code, the at least one processor may be configured to receive, from a first scan modality, a first scan that includes a set of scan images depicting metabolic information; receive, from a second scan modality, a second scan that includes a set of scan images depicting anatomical information; segment the first scan to identify a volumetric segment representing a suspected tumor, based on the depicted metabolic information; extract one or more radiomics features from the second scan, corresponding to the volumetric segment, based on the depicted anatomical information; and predict propensity of metastasis of the suspected tumor, based on the one or more radiomics features.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for predicting metastatic propensity of a tumor according to some embodiments;

FIG. 2 is a block diagram, depicting a system for predicting metastatic propensity of a tumor, according to some embodiments;

FIG. 3 is a table elaborating an example of features, selected to predict metastatic propensity of a tumor, by embodiments of the invention;

FIG. 4 is a table, elaborating exemplary statistical information that is representative of performance of a system for predicting metastatic propensity of a tumor, according to some embodiments of the invention; and

FIG. 5 is a flow diagram, depicting a method for predicting metastatic propensity of a tumor, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

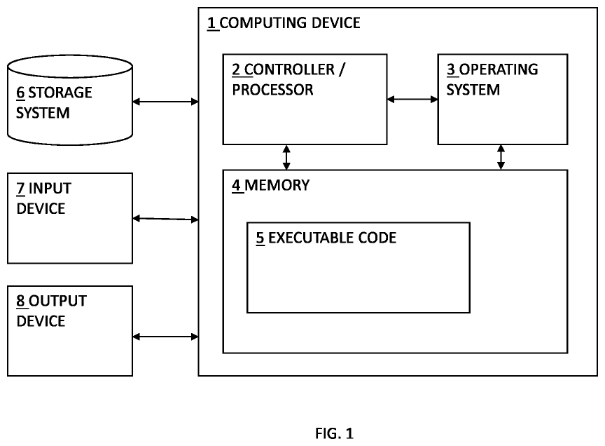

Reference is now made to FIG. 1, which is a block diagram depicting a computing device, which may be included within an embodiment of a system for predicting metastatic propensity of a tumor, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may predict metastatic propensity of a depicted tumor as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to scans of one or more scanning modalities may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPUs) or any other suitable multi-purpose or specific processors or controllers (e.g., similar to element 2), a plurality of input units, a plurality of output units, a plurality of memory units, and a plurality of storage units.

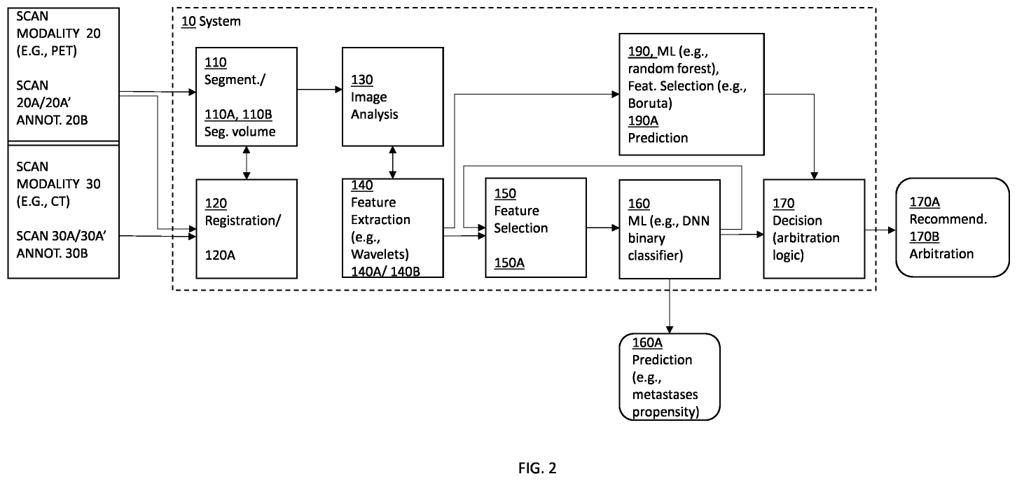

FIG. 2 is a block diagram, depicting a system 10 for predicting metastatic propensity of a tumor, according to some embodiments of the invention. System 10 may be implemented as a software module, a hardware module, or any combination thereof. For example, system 10 may be or may include a computing device such as element 1 of FIG. 1, and may be adapted to execute one or more modules of executable code (e.g., element 5 of FIG. 1) to predict metastatic propensity of a tumor, as further described herein.

As shown in FIG. 2, arrows may represent flow of one or more data elements to and from system 10 and/or among modules or elements of system 10. Some arrows have been omitted in FIG. 2 for the purpose of clarity.

According to some embodiments, system 10 may include, be associated with, or communicatively connected to one or more scanning modalities, denoted in FIG. 2 as scan modality 20 and scan modality 30.

Scan modality 20 may be, or may include a first scanning device or system, adapted to provide functional, or metabolic information pertaining to a scanned subject (e.g., a patient). Scan modality 20 may therefore be referred to herein as a metabolic scan modality 20.

For example, metabolic scan modality 20 may be, for example a Positron Emission Tomography (PET) scanning device, adapted to produce a scan data element 20A that includes a set of scan images 20A′, each depicting metabolic or functional information.

The term “metabolic” may be used in this context in a sense that scan modality 20 (e.g., PET) may depict information that represents a scanned subject's biological or chemical interaction with a predetermined material, such as a chemical tracer that may be introduced or injected to the subject as part of a medical examination, as known in the art. For example, scan modality 20 (e.g., PET) may depict information that represents intake of a Prostate-Specific Membrane Antigen (PSMA) tracer, defining a diseased volume within a prostate volume, and the metabolic feature may explicitly, or intrinsically represent or depict an amount, shape or form of PSMA intake by the diseased organ.

Additionally, or alternatively, a second scan modality 30 may be, or may include a second scanning device or system, adapted to provide anatomical information pertaining to a scanned subject (e.g., the patient). Scan modality 30 may therefore be referred to herein as an anatomic scan modality 30. For example, anatomic scan modality 30 may be a Computed Tomography (CT) scanning device, a Magnetic Resonance Imaging (MRI) scanning device and the like, adapted to produce a scan data elements 30A that include a set of scan images 30A′, each depicting anatomic or structural information, as known in the art.

As shown in FIG. 2, system 10 may include a segmentation module 110, adapted to receive one or more scan images 20A′, in a scan data element 20A of metabolic scan modality 20. According to some embodiments, segmentation module 110 may be configured to segment scan images 20A′ of scan data element 20A to identify a volumetric segment 110A representing a suspected tumor, based on the metabolic information depicted in the one or more scan images 20A′.

For example, as known the art, PET scan images 20A′ may represent metabolic information, such as localized intake of specific substances according to a grey-level legend. Expert radiologists utilize such grey-level representations to ascertain whether the depicted intake of substance represents a region or volume that is suspected as cancerous. According to some embodiments, segmentation module 110 may segment scan images 20A′ e.g., by applying a grey-level threshold on the scan images 20A′, to produce a volumetric segment 110A representing a suspected tumor.

As shown in FIG. 2, system 10 may include a registration module 120, adapted to receive from metabolic scan modality 20 one or more scan images 20A′ depicting metabolic information, and receive from anatomic scan modality 30 one or more corresponding scan images 30A′ depicting anatomical information.

According to some embodiments, registration module 120 may be configured to register, or “fuse”, as commonly referred to in the art, between scan images 20A′ and scan images 30A′ as known in the art, to produce a registered, or fused data element 120A. registered data element 120A may be, or may include a set of data elements (e.g., matrices or images) in which the functional or metabolic information of images 20A′ may be overlaid upon the anatomic information of images 30A′, in a common set of coordinates. In other words, registered data element 120A may depict a unification of the metabolic and anatomic information, where metabolic images 20A′ may be aligned according to the coordinates of anatomic images 30A′.

Additionally, or alternatively, registration module 120 may collaborate with segmentation module 110, to determine a volumetric segment 110 representing a suspected tumor in anatomic images 30A′ of anatomic scan 30A. In other words, by the process of registration, or fusion, registration module 120 may transfer volumetric segment 110A, defined by segmentation module 110 in metabolic scan 20A (e.g., PET), to a corresponding volumetric segment 110B, representing a corresponding region or volume in anatomic scan (e.g., CT) 30A.

According to some embodiments, system 10 may include a feature extraction module 140, adapted to extract one or more radiomics features 140A from anatomic scan 30A, based on the anatomical information depicted in the anatomic scan 30A. Radiomics features 140A may correspond to the volumetric segment 110B in anatomic images 30A′ of anatomic scan 30A. In other words, feature extraction module 140 may extract radiomics features 140A, representing anatomic information that is depicted in volumetric segment 110B, which in turn corresponds to a volume 110A of a suspected tumor in scan 20A.

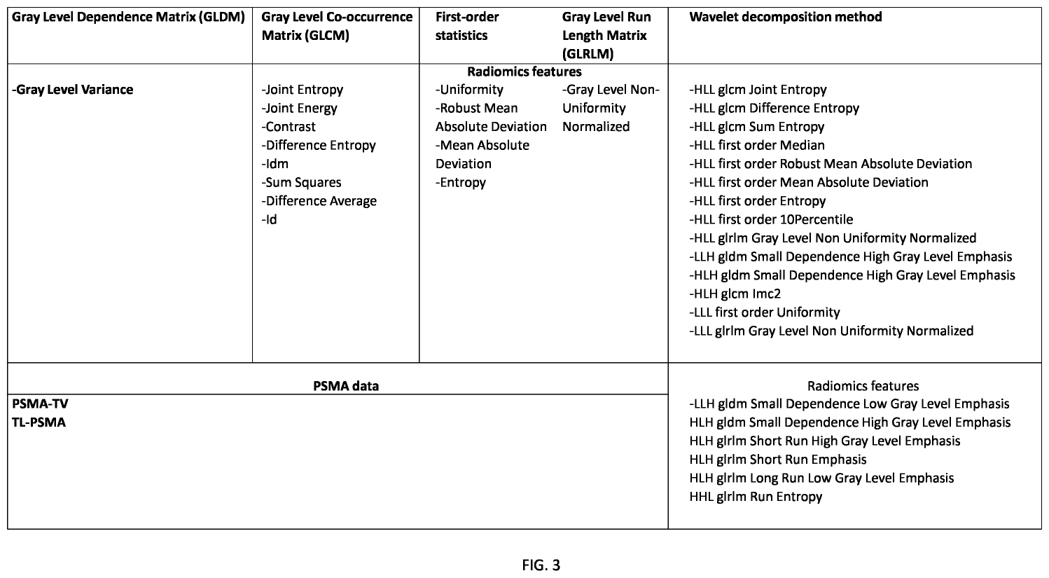

Reference is also made to FIG. 3, which is a table elaborating an example of features, selected to predict metastatic propensity of a tumor, by embodiments of the invention.

As depicted in FIG. 2, feature extraction module 140 may collaborate with an image analysis module 130, configured to apply one or more algorithms of image analysis on anatomic images 30A′ of anatomic scan 30A. Image analysis module 130 may apply the one or more algorithms of image analysis on anatomic images 30A′, to produce an initial group of radiomics features 140A.

As shown in the example of FIG. 3, image analysis module 130 may apply one or more algorithms of image analysis, that may be referred to herein as first-order statistics algorithms on anatomic images 30A′, to produce corresponding radiomics features 140A that represent first-order statistics of anatomic images 30A′.

Such first-order statistics' radiomics features 140A may include, for example, a uniformity feature 140, which may be calculated based on a sum of the squares of intensity values in anatomic images 30A′. Uniformity feature 140A may be related to as a measure of the homogeneity of the image array, where a greater uniformity implies greater homogeneity, or a smaller range of discrete intensity values.

In another example, first-order statistics' radiomics features 140A may include a robust mean absolute deviation feature 140A representing the mean distance of all intensity values from a Mean Value calculated on the subset of image array with gray levels in between, or equal to extreme percentiles (e.g., the 10th and 90th percentile).

In another example, first-order statistics' radiomics features 140A may include a mean absolute deviation feature 140A, representing the mean distance of all intensity values from the Mean Value of the image array).

In another example, first-order statistics' radiomics features 140A may include an entropy feature 140A. Entropy feature 140A may be calculated as a level of uncertainty or randomness in the image values, and may represent an average amount of information that may be required to encode the image values. Additional first-order statistics' radiomics features 140A may also be used.

Additionally, or alternatively, image analysis module 130 may apply an image analysis algorithm to compute a Grey Level Dependence Matrix (GLDM). Image analysis module 130 may subsequently calculate or extract from the GLDM matrix at least one GLDM-based radiomics feature 140A, such as a GLDM grey level variance feature 140A, expressed by the GLDM matrix.

Additionally, or alternatively, image analysis module 130 may apply an image analysis algorithm to compute a Grey Level Co-occurrence Matrix (GLCM). Image analysis module 130 may subsequently calculate or extract from the GLCM matrix at least one GLCM-based radiomics feature 140A.

Such GLCM-based radiomics features 140A may include for example a GLCM joint entropy feature 140A, a GLCM joint energy feature 140A, a GLCM difference entropy feature 140A, a GLCM contrast feature 140A, and a GLCM sum squares feature 140A.

In another example, GLCM-based radiomics features 140A may include a GLCM difference average feature 140A, which may measure a relationship between occurrences of pairs with similar intensity values and occurrences of pairs with differing intensity values of the GLCM matrix.

In another example, GLCM-based radiomics features 140A may include an Inverse Difference (ID, also referred to in the art as “Homogeneity 1”) feature, which may be regarded as another measure of local homogeneity in anatomic images 30A′: With more uniform gray levels, the denominator will remain low, resulting in a higher overall value. Additionally, or alternatively, GLCM-based radiomics features 140A may include an GLCM Inverse Difference Moment (IDM) feature 140 which, as known in the art may also be regarded as a measure of local homogeneity of an image.

Additionally, or alternatively, image analysis module 130 may apply an image analysis algorithm to compute a Grey Level Run Length Matrix (GLRLM). As known in the art, GLRLM may be used to quantify gray level runs, which are defined as the length in number of pixels, of consecutive pixels that have the same gray level value.

Image analysis module 130 may subsequently calculate or extract from the GLRLM matrix at least one GLRLM-based radiomics feature 140A. Such GLRLM-based radiomics feature 140A may include for example a GLRLM Normalized Gray Level Non-uniformity (GLNN) feature 140A which measures a similarity of gray-level intensity values in the image, where a lower GLNN value correlates with a greater similarity in intensity values.

Additionally, or alternatively, image analysis module 130 may apply one or more wavelet-based algorithms of image analysis on anatomic images 30A′ of anatomic scan 30A, to produce corresponding wavelet-based radiomics features 140A.

Such wavelet-based radiomics features 140A may include, for example an HLL first order median feature 140A (e.g., a median of a wavelet of type HLL, belonging to a first-order matrix type), an HLL first order Robust Mean Absolute Deviation feature 140A (e.g., a Robust Mean Absolute Deviation of a wavelet of type HLL, belonging to a first order matrix type), an HLL first order Mean Absolute Deviation feature 140A, an HLL first order entropy feature 140A, an HLL first order X-Percentile feature 140A, an LLL first-order uniformity feature 140A, and the like.

In another example, image analysis module 130 may apply one or more wavelet-based algorithms on the calculated GLCM matrix, to produce corresponding wavelet-based GLCM radiomics features 140A.

Such wavelet based GLCM radiomics features 140A may include, for example an HLL GLCM joint entropy feature 140A, an HLL GLCM difference entropy feature 140A, an HLL GLCM sum entropy feature 140A, and the like.

Additionally, or alternatively, image analysis module 130 may apply one or more wavelet-based algorithms on the calculated GLRLM matrix, to produce corresponding wavelet-based GLRLM radiomics features 140A.

Such wavelet based GLRLM radiomics features 140A may include, for example an HLL GLRLM Normalized Gray Level Non-uniformity feature 140A, an HLH GLRLM Short Run Emphasis feature 140A, an HLH GLRLM Short Run High Gray Level Emphasis feature 140A, an HLH GLRLM Long Run Low Gray Level Emphasis feature 140A, an HLH GLRLM Run Entropy feature 140A, and the like.

Additionally, or alternatively, image analysis module 130 may apply one or more wavelet-based algorithms on the calculated GLDM matrix, to produce corresponding wavelet-based GLDM radiomics features 140A.

Such wavelet based GLDM radiomics features 140A may include, for example an LLH GLDM Small Dependence High Gray Level Emphasis feature 140A, an HLH GLDM Small Dependence High Gray Level Emphasis feature 140A, an LLH GLDM Small Dependence Low Gray Level Emphasis feature 140A, and the like.

Additionally, or alternatively, image analysis module 130 may apply one or more algorithms of image analysis on metabolic images 20A′ of metabolic scan 20A. Image analysis module 130 may thus calculate, or produce from metabolic images 20A′ one or more image features 140B (also referred to herein as “metabolic features 140B”) that represent metabolic information represented in segmented volume 110A of metabolic images 20A′. It may be appreciated that such metabolic features 140B may be application-specific to an underlying disease and/or a specific type or instance of scan modality 20.

In the non-limiting example of FIG. 3, the underlying disease is a suspected prostate tumor. Therefore, the corresponding metabolic features 140B in this non-limiting example may include features 140B related to Prostate-Specific Membrane Antigen (PSMA) reading levels. As known in the art, PSMA (or 68 Gallium PSMA, as commonly referred to in the art) is a tracer, marked by the isotope Gallium 68, that is commonly injected to a patient during a PET procedure. The PET machine may then identify absorption of the tracer substance into specific tissues. Normal manifestation of PSMA uptake may occur in a prostate tissue, whereas abnormal manifestation of PSMA uptake (e.g., in regions other than the prostate) may indicate prostate cancer tissues.

As elaborated in the non-limiting example of FIG. 3, one such PSMA-related metabolic feature 140B may include a PSMA-Total Volume (TV) feature 140B, representing total volume of abnormal uptake within a body (thus suspected as tumors), as depicted in metabolic images 20A′ and/or anatomic images 30A′ (e.g., following registration 120).

In another example, PSMA-related metabolic features 140B may include a PSMA-Total Lesion (TL) feature 140B, representing a total number of lesions consisting of abnormal PSMA uptake within the scanned body, as depicted in metabolic images 20A′ and/or anatomic images 30A′ (e.g., following registration 120).

According to some embodiments, system 10 may include a feature selection module 150, configured to filter, or select specific radiomics features 140A of the initial, large group of radiomics features 140A, provided from feature extraction module 140, to extract a selected subset of features 150A.

As elaborated herein, system 10 may apply a machine-learning (ML) based model 160 on the selected subset of radiomics features 150A to produce a prediction 160A regarding the anatomic information and metabolic information depicted in scans 30A and 20A respectively.

For example, in some embodiments prediction 160A may include a prediction of metastatic propensity of a tumor (e.g., a prostate tumor) based, at least in part on radiomics features 140A. In this example, an anatomic scan 30A may include a CT scan of a patient, suffering from a prostate tumor, and a metabolic scan 20A may be a PET scan showing PSMA intake in the prostate. Segmented volume 110A may represent a region (e.g., in the prostate) where the suspected tumor resides in scan 20A, calculated based on the metabolic information (e.g., PSMA uptake) depicted in scan images 20A′, and segmented volume 110B may represent a corresponding region (e.g., through registration 120) in anatomic scan 30A. Radiomics features 140A may include features such as elaborated herein (e.g., in relation to FIG. 3) of the suspected segmented volume 110B of scan 30A, and the selected subset of features 150A may be provided as input to ML model 160 during an inference stage.

ML model 160 may be trained to receive radiomics features 140A (or subset 150A), and produce a prediction 160A of metastatic propensity of the suspected tumor, based on radiomics features 140A (e.g., based on radiomics features subset 150A).

Additionally, or alternatively, as elaborated herein (e.g., in relation to FIG. 3), image analysis module 130 may be configured to extract from the volumetric segment 110A metabolic scan 20A at least one metabolic feature 140B (e.g., a PSMA-related feature 140B) representing metabolic information depicted in scan 20A (e.g., PSMA intake in a prostate).

In such embodiments ML model 160 may be trained to produce a prediction 160A of propensity of metastasis of the suspected tumor based on at least one of: (a) the radiomics features 140A (or 150A) and (b) the at least one metabolic feature 140B.

According to some embodiments, ML model 160 may be trained through a supervised training process, by which “ground truth” supervisory data may be provided (e.g., by a user, via input device 7 of FIG. 1), to configure or modify ML model 160 to accurately predict 160A propensity of metastasis.

For example, during a training stage, ML model 160 may receive annotation data, representing propensity of metastasis, corresponding to metabolic scan 20A and/or anatomic scan 30A of at least one specific patient. Such supervisory data may, for example include labels or annotations (20B, 30B), attributed to one or more metabolic scans 20A and/or anatomic scans 30A of the patient. The annotations (20B, 30B) may, for example be provided by a physician, via input 7 of FIG. 1, as segments or labels in scans 20A and/or scans 30A, to reflect existence (or inexistence) and/or location of metastases, that were detected in the specific patient (e.g., in clinical real time or in hind-sight).

As known in the art, system 10 may calculate a loss function, representing a distance between prediction 160A and ground truth, as represented by the annotations 20B of metabolic scan 20A and/or annotations 30B of anatomic scan 30A. System 10 may train ML model 160 based on any known algorithm (e.g., gradient descent) to produce a prediction 160A of propensity of metastasis, based on at least one of the extracted radiomics features 140A and the at least one metabolic feature 140B, according to the annotation (20B, 30B) data.

According to some embodiments, feature selection module 150 may be configured to collaborate with ML model 160 in an iterative manner, to filter radiomics features 140A and/or metabolic features 140B, so as to select or extract feature subset 150A from features 140A/140B.

In other words, feature selection module 150 may apply a feature selection algorithm on a group of features that includes (a) radiomics features 140A and (b) the at least one metabolic feature 140B, to select a subset 150A of the group of features 140A/140B, based on the propensity of metastasis as predicted 160A by ML model 160. Feature selection module 150 may subsequently train, or retrain ML model 160 (e.g., repetitively, over time) based on the selected subset 150A of the group of features 140A and/or 140B, to produce prediction of propensity of metastasis 160A.

For example, feature selection module 150 may apply an iterative feature selection algorithm, as part of the supervised training of ML model 160. In such embodiments, during each iteration, an initial group of features that includes (a) radiomics features 140A and/or (b) metabolic features 140B may be considered. ML model 160 may be applied on the initial group of features, to produce a first prediction 160A of propensity of metastasis. Feature selection module 150 may then select a subset 150A of the group of features, and ML model 160 may be retrained, based on the selected subset 150A of features, to produce a second prediction 160A of propensity of metastasis.

The subset of features 150A may be transferred as an initial group of features for a subsequent iteration based on at least one evaluation metric pertaining to the first prediction 160A and second prediction 160A. For example, the first prediction 160A and second prediction 160A may be evaluated according to one or more types of performance metrics (e.g., accuracy, specificity, sensitivity, and the like) in relation to ground truth, as represented by the annotations 20B of metabolic scan 20A and/or annotations 30B of anatomic scan 30A. When the first prediction 160A is evaluated as inferior (e.g., lower accuracy) in relation to the second prediction 160A, then the subset of features 150A may be transferred as an initial group of features for a subsequent iteration of the feature selection algorithm, to further filter features' subset 150A. When the first prediction 160A is evaluated as superior (e.g., higher accuracy) in relation to the second prediction 160A, then a different subset of features 150A may be extracted and evaluated.

Additionally, or alternatively, the training of ML model 160 and/or selection of features 150A may be done as an iterative process, where at least one iteration, feature selection 150 module may receive and analyze a first group of features selected from (a) the radiomics features and (b) the at least one metabolic feature. Feature selection 150 module may then select a second group of features that is a subset of the first group, and system 10 may train the ML model to produce a prediction of propensity of metastasis, based on the second group of features. System 10 may then provide the second group of features to feature selection module 150 as a first group of features, for a subsequent iteration, based on propensity of metastasis as predicted by the ML model.

This iterative process may proceed until a stop condition is met. Such a stop condition may include, for example reaching a predefined performance metric value (e.g., a required accuracy, a required specificity, a required sensitivity, and the like), and/or reaching a stall in improvement of one or more performance metrics.

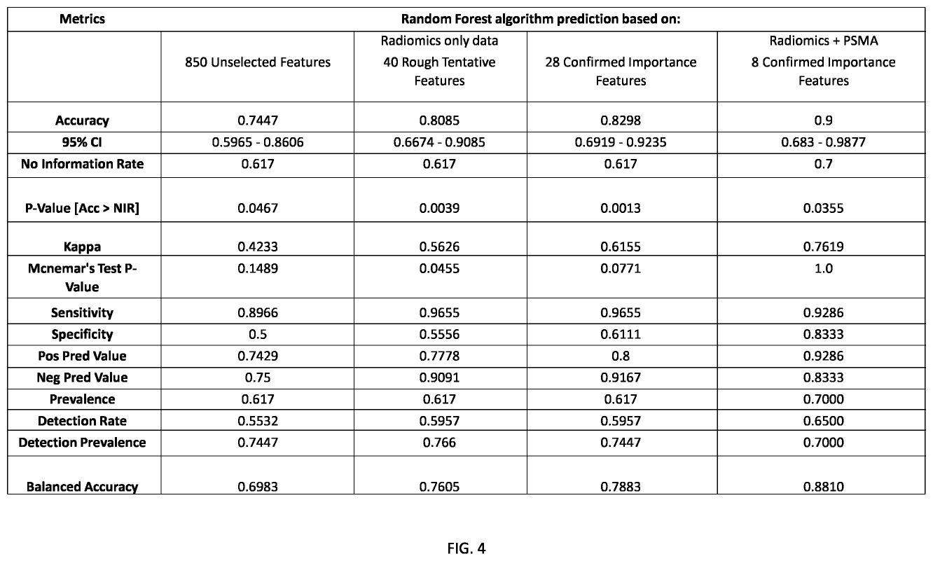

Reference is now made to FIG. 4, which is a table elaborating exemplary statistical information that has been obtained through experimentation, and is representative of performance of a system for predicting metastatic propensity of a tumor, according to some embodiments of the invention.

As shown in the example FIG. 4, the columns of the depicted table show consistent improvement in several metrics of performance (e.g., accuracy, sensitivity, specificity) as the number of utilized features is decreased (e.g., from left to right). In other words, the table of FIG. 4 demonstrates improvement of performance (e.g., prediction of metastasis propensity) following careful selection of a combination of radiomics features 140A (representing) and metabolic 140B features (representing aspects of PSMA uptake) by feature selection module.

According to some embodiments, and as depicted in FIG. 2, system 10 may include a plurality of ML models for prediction of propensity of metastasis. For example, at least one ML model may be a binary classifier ML model, such as element 160. Binary classifier ML model 160 may be trained, via a supervised training scheme, to produce a prediction of propensity of metastasis 160A based on at least one of: (a) the radiomics features and (b) the at least one metabolic feature, as elaborated herein.

Additionally, or alternatively, a second ML model may be a random forest ML model 190. Random forest ML model 190 may also be trained via a supervised training scheme, to produce a prediction of propensity of metastasis 190A based on at least one of: (a) the radiomics features 140A and (b) the at least one metabolic feature 140B, in a similar manner to that of ML model 160.

Selection of features for ML model 160 and ML model 190 may be performed separately. For example, the selection of features 150A for ML model 160 may be performed by module feature selection module 150, as elaborated herein. In contrast, the selection of features 150A for ML model 190 may be integrated within the random forest ML model 190, as known in the art.

According to some embodiments, system 10 may include a decision module 170, configured to produce at least one decision or recommendation 170A based on the prediction 160A of propensity of metastasis.

For example, decision module 170 may apply a rule-based algorithm and/or an AI based algorithm to produce a prognosis of the suspected tumor (e.g., expected rate of metastases) based on at least one of: predicted propensity of metastasis 160A, features' subset 150A, radiomics features 140A, and/or metabolic features 140B.

In another example, decision module 170 may apply a rule-based algorithm and/or an AI based algorithm to recommend a course of treatment for the suspected tumor, based on at least one of metastasis 160A, features' subset 150A, radiomics features 140A, and/or metabolic features 140B.

Additionally, or alternatively, decision module 170 may be configured to arbitrate between prediction of propensity 160A/190A of metastasis of both ML models (160/190 respectively), and produce a unified prediction 170B of propensity of metastasis of the suspected tumor, based on the arbitration.

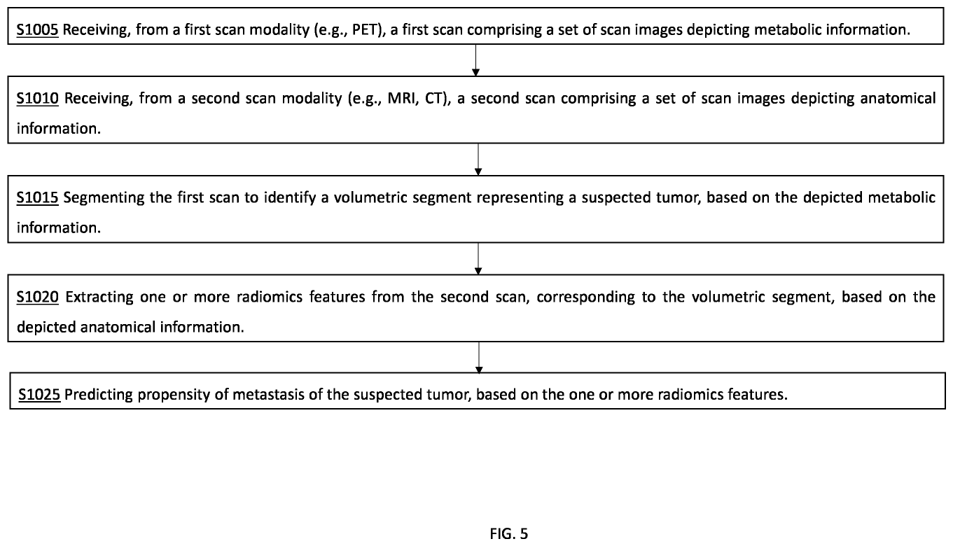

Reference is now made to FIG. 5, which is a flow diagram, depicting a method for predicting metastatic propensity of a tumor, by at least one processor (e.g., processor 2 of FIG. 1), according to some embodiments.

As shown in steps S1005 and S1010, the at least one processor 2 may receive (e.g., via input 7 of FIG. 1) from a first scan modality 20, a first scan 20A that may include a set of scan images 20A′ depicting metabolic information, and from a second scan modality 30 a second scan 30A may include a set of scan images 30A′ depicting anatomical information.

As shown in step S1015, the at least one processor 2 may segment the first scan 20A to identify a volumetric segment 110A representing a suspected tumor, based on the depicted metabolic information (e.g., PSMA uptake).

As shown in step S1020, the at least one processor 2 may extract one or more radiomics features 140A from the second scan 30A, corresponding to the volumetric segment 110A, based on the depicted anatomical information.

As shown in step S1025, the at least one processor 2 may employ one or more ML models (e.g., binary classifier ML model 160, random forest ML model 190) to predict (160A, 190A, 170B) a propensity of metastasis of the suspected tumor, based at least in part on the one or more radiomics features 140A.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of predicting propensity of metastasis of a tumor in a patient by at least one processor, the method comprising:
receiving, from a first scan modality, a first scan comprising a set of scan images depicting metabolic information;
receiving, from a second scan modality, a second scan comprising a set of scan images depicting anatomical information;
segmenting the first scan to identify a volumetric segment representing a suspected tumor, based on the depicted metabolic information;
extracting one or more radiomics features from the second scan, corresponding to the volumetric segment, based on the depicted anatomical information; and
predicting propensity of metastasis of the suspected tumor, based on the one or more radiomics features.

2. The method of claim 1, further comprising determining at least one of a prognosis and a suggested course of treatment for the suspected tumor, based on the predicted propensity of metastasis.

3. The method of claim 1, wherein the second scan modality is a Positron Emission Tomography (PET) scan modality, and wherein the first scan modality is selected from a Computed Tomography (CT) scan modality, and a Magnetic Resonance Imaging (MRI) scan modality.

4. The method of claim 1, wherein predicting propensity of metastasis of the suspected tumor comprises applying at least one machine-learning (ML) model on the extracted radiomics features, to produce a prediction of propensity of metastasis.

5. The method of claim 4, further comprising:
extracting, from the volumetric segment of the first scan, at least one metabolic feature representing the depicted metabolic information; and
training the at least one ML model to produce a prediction of propensity of metastasis, based on at least one of: (a) the radiomics features and (b) the at least one metabolic feature.

6. The method of claim 5, further comprising:
applying a feature selection algorithm on a group of features comprising (a) the radiomics features and (b) the at least one metabolic feature, to select a subset of the group of features, based on the propensity of metastasis as predicted by the at least one ML model; and
further training the at least one ML model based on the subset of the group of features, to produce a prediction of propensity of metastasis.

7. The method of claim 5, further comprising training the at least one ML model in an iterative process, wherein at least one iteration of the iterative process comprises:
receiving a first group of features selected from (a) the radiomics features and (b) the at least one metabolic feature;
selecting a second group of features that is a subset of the first group;
training the at least one ML model to produce a prediction of propensity of metastasis, based on the second group of features; and
providing the second group of features as a first group of features for a subsequent iteration, based on propensity of metastasis as predicted by the at least one ML model.

8. The method of claim 4, further comprising:
extracting at least one metabolic feature, representing the metabolic information depicted in the volumetric segment of the first scan;
receiving annotation data, representing propensity of metastasis, corresponding to at least one of the first scan and second scan; and
training the at least one ML model to produce a prediction of propensity of metastasis, based on at least one of the extracted radiomics features and the at least one metabolic feature, according to the annotation data.

9. The method of claim 1, wherein extracting the radiomics features comprises:
computing a Grey Level Co-occurrence Matrix (GLCM), based on at least one image of the second scan; and
calculating at least one GLCM-based radiomics feature selected from a list consisting of a GLCM joint entropy feature, a GLCM joint energy feature, a GLCM difference entropy feature, a GLCM contrast feature, a GLCM sum squares feature, a GLCM difference average feature, a GLCM Inverse Difference feature, and a GLCM Inverse Difference Moment IDM feature.

10. The method of claim 9, wherein extracting the radiomics features comprises applying one or more wavelet-based algorithms on the GLCM matrix, to produce at least one wavelet-based GLCM radiomics feature, selected from a list consisting of an HLL GLCM joint entropy feature, an HLL GLCM difference entropy feature, and an HLL GLCM sum entropy feature.

11. The method of claim 1, wherein extracting the radiomics features comprises:
computing a Grey Level Run Length Matrix (GLRLM), based on at least one image of the second scan; and
calculating a Normalized Gray Level Non-uniformity (GLNN) radiomics feature based on the GLRLM matrix.

12. The method of claim 11, wherein extracting the radiomics features comprises applying one or more wavelet-based algorithms on the GLRLM matrix, to produce at least one wavelet-based GLRLM radiomics feature, selected from a list consisting of an HLL GLRLM Normalized Gray Level Non-uniformity feature, an HLH GLRLM Short Run Emphasis feature, an HLH GLRLM Short Run High Gray Level Emphasis feature, an HLH GLRLM Long Run Low Gray Level Emphasis feature, and an HLH GLRLM Run Entropy feature.

13. The method of claim 1, wherein extracting the radiomics features comprises computing a wavelet-based radiomics feature, based on at least one image of the second scan, wherein said wavelet-based radiomics feature is selected from a list consisting of an HLL first order median feature, an HLL first order Robust Mean Absolute Deviation feature, an HLL first order Mean Absolute Deviation feature, an HLL first order entropy feature, an HLL first order X-Percentile feature, and an LLL first-order uniformity feature.

14. The method of claim 1, wherein extracting the radiomics features comprises
computing a Grey Level Dependence Matrix (GLDM), based on at least one image of the second scan; and
calculating at least one GLDM-based radiomics feature based on the GLDM matrix, said GLDM-based radiomics feature selected from a list consisting of a GLDM grey level variance feature.

15. The method of claim 14, further comprising applying one or more wavelet-based algorithms on the calculated GLDM matrix, to produce corresponding wavelet-based GLDM radiomics features, selected from a list consisting of an LLH GLDM Small Dependence High Gray Level Emphasis feature, an HLH GLDM Small Dependence High Gray Level Emphasis feature, and an LLH GLDM Small Dependence Low Gray Level Emphasis feature.

16. The method of claim 5, wherein the at least one ML model comprises a first ML model that is a binary classifier, and a second ML model that is a random forest ML model.

17. The method of claim 16, wherein both the binary classifiers and the random forest ML models are trained to produce a prediction of propensity of metastasis, based on at least one of: (a) the radiomics features and (b) the at least one metabolic feature, and wherein the method further comprises:
arbitrating between prediction of propensity of metastasis of both ML models; and
producing a prediction of propensity of metastasis of the suspected tumor, based on the arbitration.

18. The method of claim 1, wherein the volumetric segment is comprised within a prostate of the patient, and wherein the metabolic information represents a depicted intake of Prostate-Specific Membrane Antigen (PSMA) within the prostate.

19. A system for predicting propensity of metastasis of a tumor, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive, from a first scan modality, a first scan comprising a set of scan images depicting metabolic information;
receive, from a second scan modality, a second scan comprising a set of scan images depicting anatomical information;
segment the first scan to identify a volumetric segment representing a suspected tumor, based on the depicted metabolic information;
extract one or more radiomics features from the second scan, corresponding to the volumetric segment, based on the depicted anatomical information; and
predict propensity of metastasis of the suspected tumor, based on the one or more radiomics features.
